(19) United States
(12) Patent Application Publication
(10) Pub. No.: US 2012/0016136 A1
(43) Pub. Date: Jan. 19, 2012

(54) PROCESS FOR THE PREPARATION OF PROSTAGLANDIN DERIVATIVES

(76) Inventors: Giancarlo Biffi, Milano (MI) (IT); Alessandro D'Alfonso, Pavia (PV) (IT); Lazzaro Feliciani, Milano (MI) (IT); Alessio Porta, Pavia (PV) (IT); Giovanni Vidari, Pavia (PV) (IT); Enrico Viscardi, Milano (MI) (IT); Giuseppe Zanoni, Pavia (PV) (IT)

(21) Appl. No.: 13/203,670

(22) PCT Filed: Feb. 18, 2010
(86) PCT No.: PCT/IB2010/000315

§ 371 Date: Sep. 29, 2011

(30) Foreign Application Priority Data
Feb. 27, 2009 (IT) MI2009A000292

(51) Int. Cl. C07F 7/18; C07C 67/08; C07C 231/14

(52) U.S. Cl. 549/214; 564/171; 560/60; 556/441; 556/419

(57) ABSTRACT

The invention concerns a new process for the preparation of prostaglandin derivatives, in particular prostaglandin F_{2α} derivatives, for example bimatoprost, latanoprost and travoprost, the new intermediates of said process and their use in the preparation of prostagblandin derivatives.

### SUMMARY OF THE INVENTION

The present invention concerns a new process for the preparation of prostaglandin derivatives, in particular prostaglandin F_{2α} derivatives, for example bimatoprost, latanoprost and travoprost. The invention also concerns the new intermediates of said process and their use in the preparation of prostaglandin derivatives.

### TECHNICAL BACKGROUND

Prostaglandins are a class of endogenous molecules derived from arachidonic acid by action of prostaglandin synthetase and have various biological activities.

Structurally, prostaglandins are formed of a ring and two side chains, said ring and chains being replaceable (usually by hydroxy or keto groups) and optionally being partly unsaturated.

The compounds bimatoprost, latanoprost and travoprost (DCI) are analogues of prostaglandin F_{2α} and are used in therapy in the treatment of glaucoma, in particular to reduce high endo-ocular pressure.

The derivatives of the prostaglandins like those mentioned above are usually prepared according to a synthesis method which starts from Corey aldehyde ([3α(3αα,4α,5β,6αα)]-(−)-5-(hydroxy)hexahydro-2-oxo-2H-cyclopenta[b]furan-4-carboxaldehyde), hydroxy-protected, to which the two side chains are attached.

The protection of the hydroxy group is generally obtained via the formation of esters, using for example benzoic acid or its derivatives or aliphatic carboxylic acids such as acetic acid, or with THP (tetrahydropyranyl). The protection by means of the protective groups indicated above has considerable drawbacks, for example the difficulty of final release, not facilitating the subsequent asymmetric synthesis steps or, in the case of the THP, the introduction of a further chiral centre which entails the formation of diastereoisomers, significantly complicating the NMR spectra and the chromatographic profile.

Syntheses of prostaglandin derivatives which use TBS (tert-butyldimethylsilyl) as the protective group for the hydroxy group of the Corey aldehyde are known. However, these syntheses result in end products with very poor yields.

### DESCRIPTION OF THE INVENTION

A new synthesis process has now been found which, starting from the Corey aldehyde protected with TBS, produces prostaglandin derivatives, in particular prostaglandin F_{2α} derivatives, with excellent yields.

Thus, according to one of its aspects, the invention concerns a process for the preparation of prostaglandin derivatives which comprises:
a) reacting the ([3αR(3αα,4α,5β,6αα)]-(−)-5-(tert-butyldimethylsilyl)hexahydro-2-oxo-2H-cyclopenta[b]furan-4-carboxaldehyde) of formula (I)

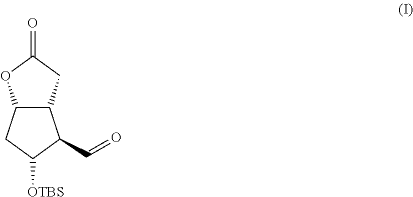

with a phosphonate of formula (II)

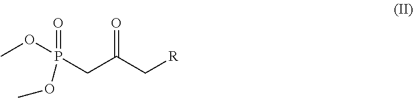

in which R represents a benzyl group or a phenoxy group, in the latter case the phenyl can be optionally substituted by a group selected from halogens, hydroxy derivatives, alkyls, aryls, heteroaryls and trifluoromethyl,
in the presence of a base and an appropriate solvent, to give the compound of formula (III)

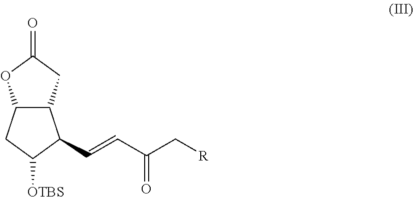

and
b) reducing the keto group of the side chain with an asymmetric reducing agent in the presence of an appropriate solvent, to give the compound (IV)

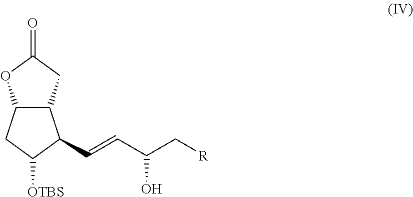

The ([3αR(3αα,4α,5β,6αα)]-(−)-5-(tert-butyldimethylsilyl)hexahydro-2-oxo-2H-cyclopenta[b]furan-4-carboxaldehyde) of formula (I), indicated hereinafter also as “Corey-TBS aldehyde”, and the compound of formula (II) are molecules known and available on the market.

The term “hydroxy derivatives” indicates a hydroxy or structurally correlated groups of formula O—X, where X is an alkyl or an aryl; a preferred hydroxy derivative is OH.

The term “alkyls” indicates linear or branched alkyls, saturated or unsaturated, C1-C10, preferably C1-C4.

The term “aryls” includes for example the phenyl group, phenyls substituted, preferably with the trifluoromethyl or fluorine group.

The term “heteroaryls” includes for example imidazoles, indols, pyridines, furans and thiophenes, optionally substituted.

The term “halogen” refers to an atom of bromine, chlorine, fluorine or iodine, the fluorine being preferred.

According to a preferred embodiment of the invention, R represents a benzyl group.

According to a further embodiment of the invention, R represents a phenoxy group substituted with a trifluoromethyl group, advantageously R is a 3-trifluoromethyl phenoxy.

The base used in the reaction step (a) is a strong base, such as a hydride or an alcoholate of alkaline metals, preferably hydride, for example sodium hydride.

The solvent used in step (a) is advantageously an inert solvent, for example an ether, such as dimethoxyethane or a cyclic ether, like tetrahydrofuran or 2-methyl tetrahydrofuran, the latter cyclic ethers being preferred.

The reactions of the steps (a) and (b) are preferably carried out in an inert atmosphere, for example under argon or nitrogen.

“Asymmetric reduction agent” here indicates a reducing agent able to reduce the ketone group of the side chain to a hydroxy group, approaching the re face of the carbonylic system. Said reducing agent is advantageously DIP-Cl (diisopinocamphenylchlorine borane).

Both steps (a) and (b) above are carried out at low temperatures, for example between −30° and +10° C., advantageously between −30° C. and 0° C. Details of more advantageous reaction conditions are provided in the experimental section of the present invention.

The compounds of formula (III) and (IV) indicated above are new compounds and constitute a further subject-matterof the present invention.

In particular, the compound of formula (IV) represents a key new intermediate in the synthesis of prostaglandin derivatives, especially prostaglandin F_{2α} derivatives.

The compounds of formula (IV) in which R is a non-substituted benzyl group or a phenoxy group substituted with a trifluoromethyl group, advantageously the 3-trifluoromethylphenoxy group, are particularly preferred compounds.

Thus according to another of its embodiments, the invention concerns use of the compound of formula (IV) as an intermediate for the synthesis of prostaglandin derivatives, for example of prostaglandin F_{2α} such as bimatoprost, latanoprost and travoprost.

The compound of formula (IV) in which R is a benzyl group is preferably prepared according to the following Scheme (I):

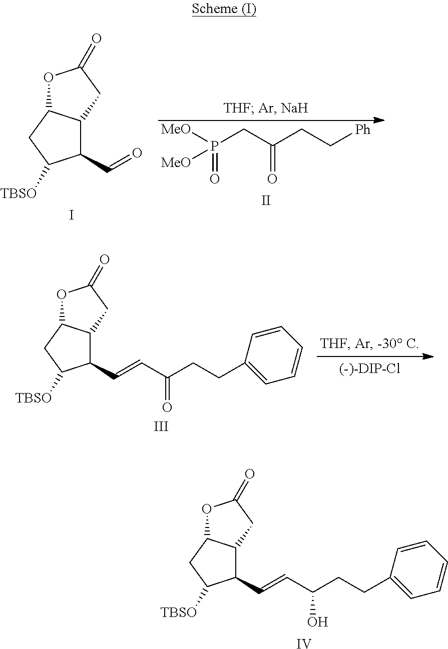

The meanings of the codes used in the schemes are given in the experimental section that follows.

According to another of its embodiments, the invention concerns a process for preparation of the bimatoprost which comprises:
(c) protecting the free hydroxy of the compound of formula (IV) in which R is a non-substituted benzyl group to give the compound of formula (V)

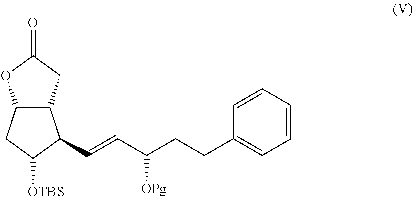

where Pg is a protective group, preferably TBS;
(d) reducing the keto group to give the compound of formula (VI)

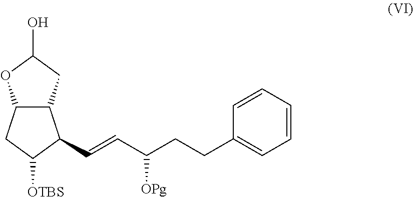

(e) reacting the compound of formula (VI) with the compound of formula:

Ph₃P═CH(CH)₃COOM
where M is an alkaline metal, preferably potassium, to give the compound of formula (VII)

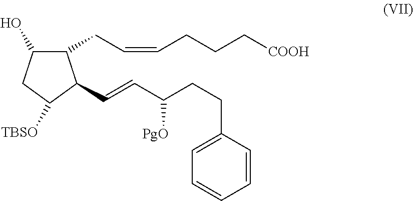

(f) esterifying the compound of formula (VII) to give the compound (VIII)

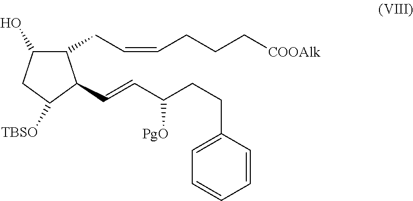

where Alk is the residue of an inferior alkyl, preferably a C1-C4 alkyl, for example methyl;
(g) forming the amide of the compound (VIII) to give the compound (IX)

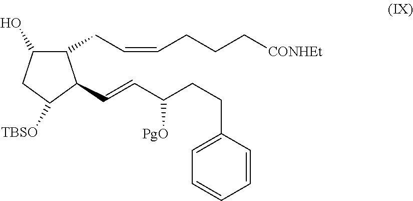

and
(h) cleaving the compound (IX) from the protective groups to give the bimatoprost of formula (X)

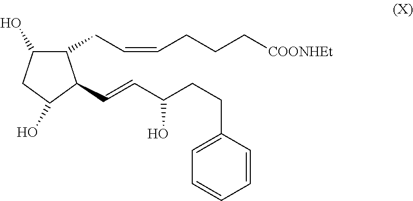

According to a particularly preferred embodiment, the process for the preparation of the bimatoprost is performed according to the following Scheme (II)

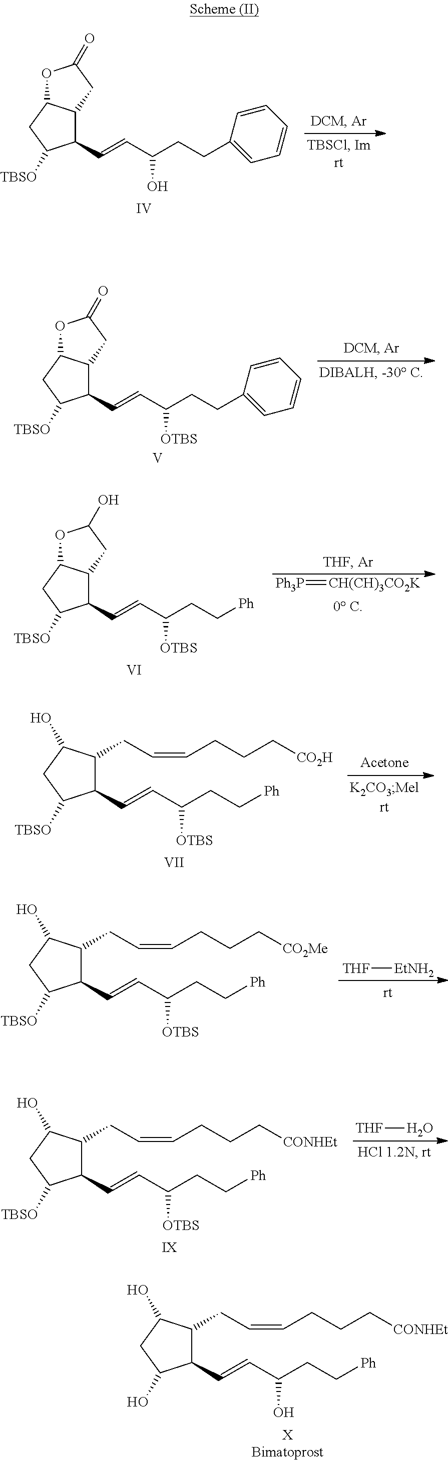

According to another of its embodiments, the invention concerns a process for preparation of the latanoprost which comprises:
(c′) reducing the double bond of the compound of formula (IV) in which R is a non-substituted benzyl group to give the compound of formula (XI)

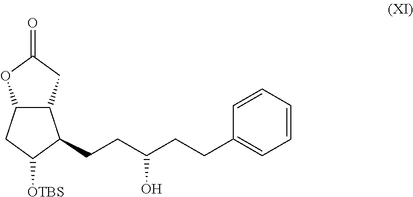

(d′) protecting the free hydroxy group of the compound of formula (XI) to give the compound of formula (XII) where Pg is a protective group, preferably TBS

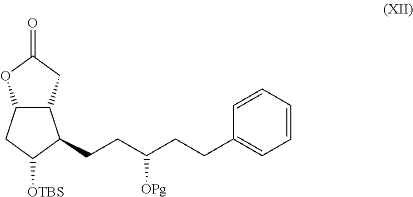

(e′) reducing the keto group of the compound of formula (XII) to give the compound of formula (XIII)

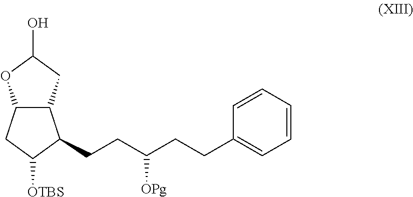

(f′) reacting the compound of formula (XIII) with the compound of formula:

Ph₃P═CH(CH)₃COOM
where M is an alkaline metal, preferably potassium, to give the compound of formula (XIV)

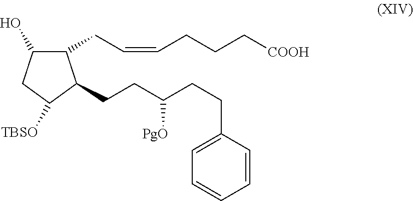

(g′) deprotecting the compound of formula (XIV) to give the compound (XV)

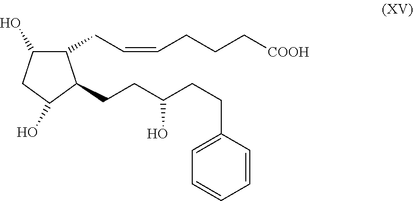

and
(h′) esterifying the compound (XV) to give the latanoprost of formula (XVI)

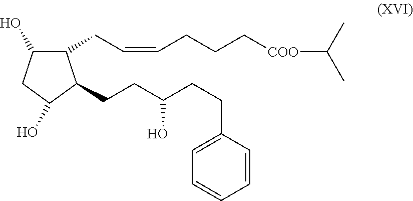

According to a particularly preferred embodiment, the process for the preparation of the latanoprost is performed according to the following Scheme (III)

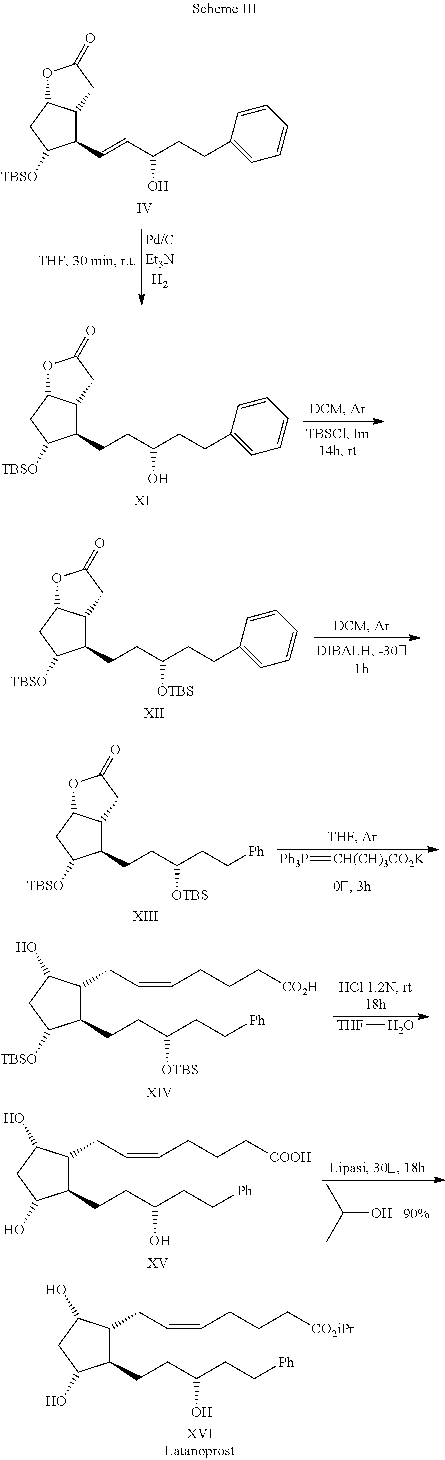

According to another of its embodiments, the invention concerns a process for preparation of the travoprost which comprises:
(c″) protecting the free hydroxy of the compound of formula (IV) in which R is a 3-trifluoromethylphenoxy group to give the compound of formula (XVII)

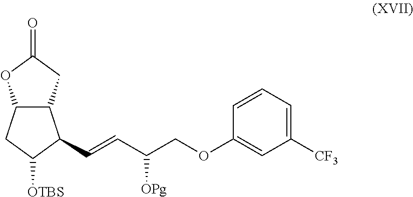

where Pg is a protective group, preferably TBS;
(d″) reducing the keto group to give the compound of formula (XVIII)

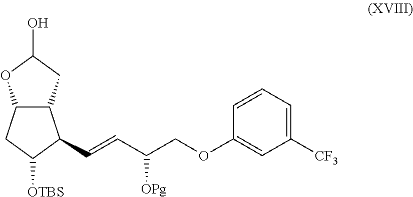

(e″) reacting the compound of formula (XVIII) with the compound of formula:

Ph₃P═CH(CH)₃COOM
where M is an alkaline metal, preferably potassium, to give the compound of formula (XIX)

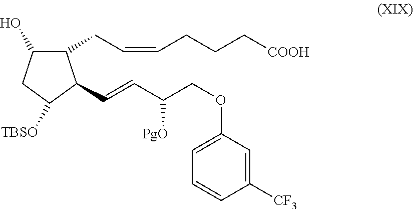

(f′) deprotecting the compound of formula (XIX) to give the compound (XX)

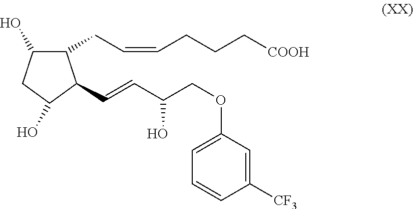

(g″) esterifying the compound (XX) to give the travoprost of formula (XXI)

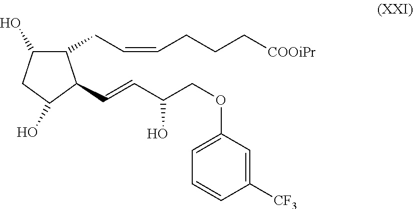

where iPr is an isopropylic residue.

According to a particularly preferred embodiment, the process for the preparation of the travoprost is performed according to the following Scheme (IV)

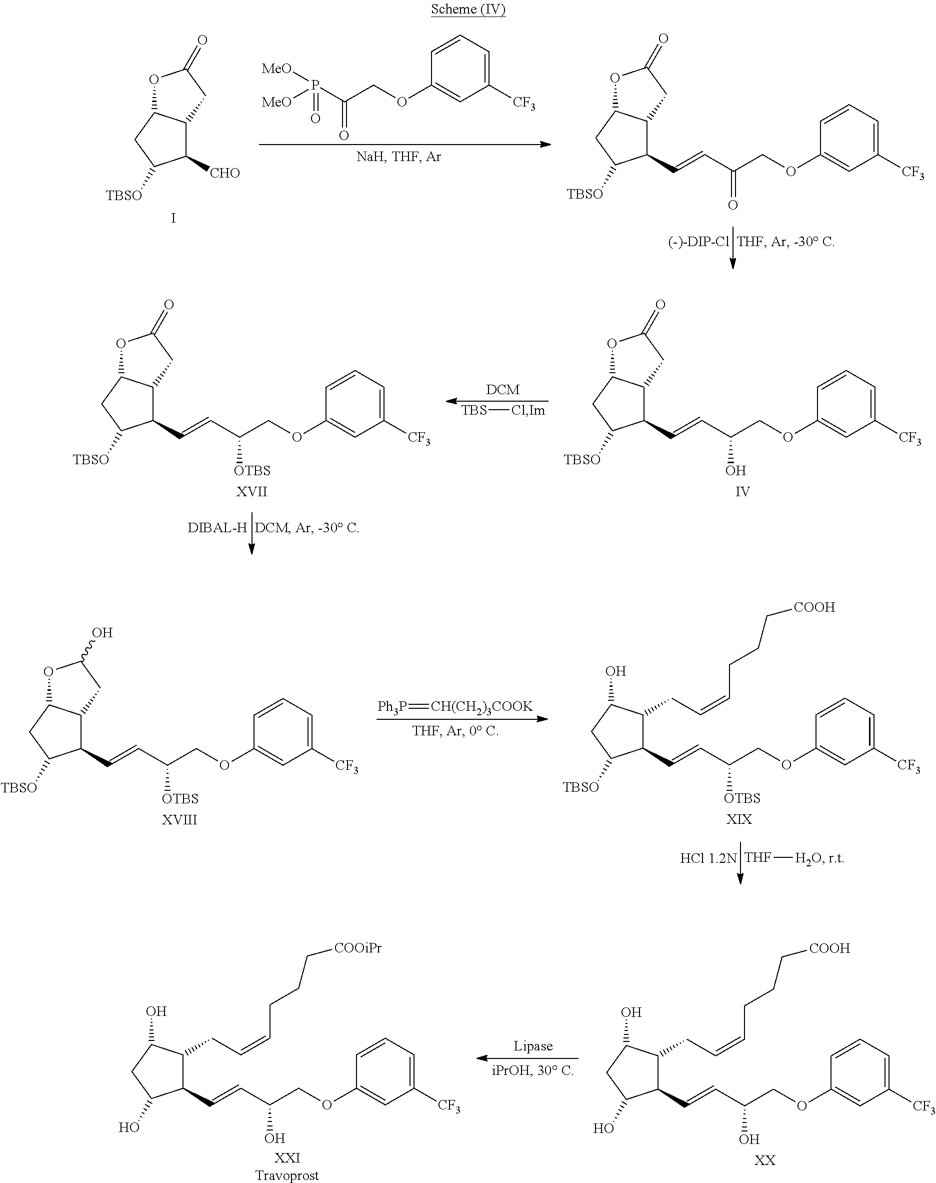

Details relative to the syntheses described above are provided in the experimental section of the present description.

The compounds selected independently from the compounds of formula (V), (VI), (VII), (IX), (XI), (XII), (XIII), (XIV), (XV), (XVII), (XVIII), (XIX) and (XX) as defined above are new intermediates and constitute, each one independently, further subject-matterof the present invention. Said compounds in which Pg, when present, designates a TBS group are particularly preferred. Even more preferred are said compounds in which Pg, when present, designates a TBS group and in which R, when present, is a non-substituted benzyl group or a phenoxy group substituted with a trifluoromethyl group, advantageously 3-trifluoromethyl.

The invention also concerns the compounds bimatoprost, latanoprost and travoprost obtained with the process of the invention.

### EXPERIMENTAL SECTION

### Example 1

### Preparation of the Key Intermediate of General Formula (Iv) where R is a Benzyl Residue

### Preparation of the Compound III (Scheme I)

A solution of dimethyl-(2-oxo-4-phenylbutyl)phosphonate (9.72 g, 0.038 moles, 1.08 eq) in tetrahydrofuran (340 mL) is slowly added to a suspension of NaH (60% in weight in mineral oil, 1.46 g, 0.036 moles, 1.04 eq) in tetrahydrofuran (200 mL) cooled to 0° C., in a static argon atmosphere. After the additions, the previously milky solution becomes clear, the ice and water bath is removed, and the solution is left under vigorous stirring at room temperature for one hour during which the formation of a white precipitate is observed. After one hour the solution is brought back to 0° C. and Corey I aldehyde is added (10 g, 0.035 moles) dissolved in tetrahydrofuran (75 mL), after which the ice bath is removed. After 90 minutes the reaction is complete and to quench it the following are added: acetic acid (2 mL), a saturated solution of ammonium chloride (230 mL), saline solution (150 mL) and water (50 mL); after stirring for a few minutes the phases are separated. The aqueous phase is extracted with AcOEt. The re-combined organic phases are dried on MgSO₄, the solid residue is filtered and the solvent is removed at reduced pressure. The product is purified by means of column chromatography (hexane-AcOEt 8:2 v/v). The pure product is obtained as a colourless oil with a yield of 93%.

### Preparation of the Compound Iv

The (−)-DIP-Cl (50-65% in weight in heptane, 55 ml, 0.14 moles, 6 eq) is added under stirring to a solution of III (10 g, 0.024 moles) in tetrahydrofuran (110 mL), in a static argon atmosphere at a temperature of −30° C.; the colourless transparent solution becomes clear pale yellow and over time this colouring disappears. After 5 hours at −25° C. the reaction is complete; sodium bicarbonate (35 g) and methanol (58 mL) are added, then the solution is left under stirring at room temperature for 10 hours, after which water is added (80 mL) and the solution is diluted with AcOEt, the phases are separated and the aqueous phase is extracted with AcOEt; the re-combined organic phases are dried on magnesium sulphate, the solid residue is filtered and the solvent is removed at reduced pressure. The product is purified by means of column chromatography (hexane-AcOEt 8:2, v/v). The pure product is obtained as a colourless oil with a yield of 85%.

### Example 2

### Preparation of the Key Intermediate of General Formula (Iv) where R is a 3-trifluoromethylphenoxy residue

### Preparation of the Compound III (Scheme Iv)

A solution of [2-oxo-3-(3-trifluoromethyl-phenoxy)-propyl]-phosphonic acid dimethyl ester (489 mg, 1.5 mmoles, 1.2 eq) in tetrahydrofuran (10 mL) is slowly added to a suspension of NaH (60% in weight in mineral oil, 55 mg, 1.37 mmoles, 1.1 eq) in tetrahydrofuran (6 mL) cooled to 0° C., in a static argon atmosphere. After the additions, the previously milky solution becomes clear, the ice and water bath is removed and the solution is left under vigorous stirring at room temperature for one hour during which the formation of a white precipitate is observed. After one hour the solution is brought back to 0° C. and the Corey I aldehyde (355 mg, 1.25 mmoles) is added dissolved in tetrahydrofuran (3 mL); after which the ice bath is removed. After 90 minutes the reaction is complete and to quench it, the following are added: acetic acid (70 μL), a saturated solution of ammonium chloride (20 mL), brine (15 mL) and water (10 mL); after stirring for a few minutes, the phases are separated. The aqueous phase is extracted with AcOEt. The re-combined organic phases are dried on MgSO₄, the solid residue is filtered and the solvent is removed at reduced pressure. The product is purified by means of column chromatography (hexane-AcOEt 8:2 v/v). The pure product is obtained as a white solid with a yield of 85%.

### Preparation of the Compound Iv

The (−)-DIP-Cl (50-65% in weight in heptane, 1.22 ml, 3.17 moles, 6 eq) is added under stirring to a solution of III (265 mg, 0.529 mmoles) in tetrahydrofuran (5 mL), in a static argon atmosphere at a temperature of −30° C.; the colourless transparent solution becomes clear pale yellow and over time this colouring disappears. After 5 hours at −25° C. the reaction is complete; sodium bicarbonate (450 mg) and methanol (800 μL) are added, then the solution is left under stirring at room temperature for 10 hours, after which water is added (5 mL) and the solution is diluted with AcOEt, the phases are separated and the aqueous phase is extracted with AcOEt; the re-combined organic phases are dried on magnesium sulphate, the solid residue is filtered and the solvent is removed at reduced pressure. The product is purified by means of column chromatography (hexane-AcOEt 8:2, v/v). The pure product is obtained as a colourless oil with a yield of 93%.

### Example 3

### Preparation of the Bimatoprost (Scheme II)

### Preparation of the Compound V

Imidazole (720 mg, 10.6 mmoles, 2.5 eq) and TBS-Cl (702 mg, 4.67, 1.1 eq) are added at room temperature in the above order to a solution of alcohol IV (1.76 g, 4.24 mmoles) in dichloromethane (35 mL) and the formation of a white precipitate is immediately noted; the reaction proceeds under stirring at room temperature and is complete after 18 hours; to quench it, a saturated solution of sodium bicarbonate (30 mL) is added, it is diluted with dichloromethane (25 mL), the phases are separated, the aqueous phase is extracted with dichloromethane, the re-combined organic phases are dried on magnesium sulphate and filtered, and lastly the solvent is removed at reduced pressure. The product is purified by means of column chromatography (hexane-AcOEt 9:1 v/v). The pure product is obtained as a white solid with a yield of 95%.

### Preparation of the Compound VI

DIBAL-H (1 M in hexane, 4.12 ml, 4.12 mmoles, 1.15 eq) is slowly added to a solution of lactone V (1.9 g, 3.58 mmoles) in dichloromethane (60 mL) cooled to −30° C. in a static argon atmosphere. After the additions have been made, the reaction is complete after 30 min. To decompose the reducing agent, a saturated solution of Rochelle salts (80 mL) is added, again at −30° C., and the solution is diluted with dichloromethane; after a few minutes the dry ice and acetone bath is removed and the solution is left under vigorous stirring until the two phases can be clearly distinguished (approximately 90 minutes). The phases are separated and the aqueous phase is extracted with dichloromethane; the re-combined organic phases are dried on magnesium sulphate, the solid residue is filtered and the solvent is removed at reduced pressure. The product obtained with a quantitative yield is not purified but used directly for the subsequent reaction.

### Preparation of the Compound VII

The potassium tert-butylate (4.5 g, 32.2 mmoles, 9 eq) is added at room temperature in small portions to a suspension of (4-carboxybutyl)triphenylphosphonium bromide (9 g, 16.1 mmoles, 4.5 eq) in tetrahydrofuran (45 mL) in a static argon atmosphere; during the addition the solution heats up and takes on an orange colouring which increasingly verges on bright red. The solution is left under stirring for 30 minutes at room temperature and is then cooled to 0° C., after which the lactol VI is added ‘via cannula’ (1.9 g, 3.57 mmoles) dissolved in tetrahydrofuran (20 mL); the solution turns paler, after 15 minutes the ice and water bath is removed and the solution is left under stirring at room temperature. After three hours the reaction is complete and is quenched by adding a saturated solution of ammonium chloride (100 mL) and acetic acid (1.9 mL, 1.05 eq with respect to the potassium tert-butylate). The solution is left under stirring for 15 minutes and is then diluted with ethyl ether, the phases are separated, the aqueous phase is extracted with ethyl ether and the re-combined organic phases are dried on magnesium sulphate, filtered and concentrated at reduced pressure. The compound VII is obtained which is used directly in the subsequent reaction.

### Preparation of the Compound VIII

Potassium carbonate (2.18 g, 15.8 mmoles, 5 eq) and MeI (2.9 mL, 47.5 mmoles, 15 eq) are added to a solution of the compound VII (1.9 g, 3.17 mmoles) in acetone (45 mL) at room temperature. After a few minutes the formation of a white precipitate is noted and the reaction is complete after approximately 18 hours under vigorous stirring at room temperature. The solution is diluted with ethyl ether (30 mL) to promote the precipitation of salts and is then filtered. The solvent is removed at reduced pressure and is then recovered with ethyl ether (60 mL) and water (50 mL), the phases are separated, the aqueous phase is extracted with ethyl ether, the re-combined organic phases are dried on magnesium sulphate, filtered and lastly the solvent is removed at reduced pressure. The product is purified by means of column chromatography (hexane-AcOEt 9:1 v/v). The compound VIII is obtained as a colourless oil with a yield of 92%.

### Preparation of the Compound IX

Ethyl amine (70% in water, 60 mL) is added at room temperature to a solution of the compound VIII (1.25 g, 0.002 moles) in tetrahydrofuran (12 mL), the reaction is performed at this temperature under magnetic stirring and is complete after approximately 52 hours. The reaction is quenched by cooling the solution to 0° C. and adding in small portions a 15 M solution of NaHSO₄ until a pH of approximately 6 is measured, then phosphate buffer is added (pH=6.8, 50 mL) and ethyl ether (80 mL), the two phases are separated, the aqueous phase is extracted with ethyl ether, the re-combined organic phases are washed with brine, dried on magnesium sulphate and filtered, and then the solvent is removed at reduced pressure. The products are purified by means of column chromatography (hexane-AcOEt 8:2 v/v). The compound IX is obtained with a yield of 90%.

### Preparation of the Compound X (Bimatoprost)

HCl 1.2 N (2 mL) is added at room temperature to a solution of the compound IX (950 mg, 1.5 mmoles) in a tetrahydrofuran/water 1:1 (50 mL) mixture, the reaction is performed under vigorous stirring and is complete after approximately 18 hours. It is quenched by adding phosphate buffer (pH=6.8, 150 mL), then the organic phase is diluted with AcOEt, the two phases are separated, the aqueous phase is extracted with AcOEt, the re-combined organic phases are dried on magnesium sulphate and filtered, and lastly the solvent is removed at reduced pressure. The product is purified by means of column chromatography (AcOEt-methanol 95:5 v/v). The Bimatoprost is obtained pure as a colourless oil with a yield of 91%.

### Example 4

### Preparation of the Latanoprost (Scheme III)

### Preparation of the Compound XI

Triethylamine (4.3 mL, 0.031 moles, 10 eq) and palladium catalyst 10% on carbon (130 mg, 10% in weight with respect to 3) are added to a solution of IV in tetrahydrofuran (50 mL), three vacuum-hydrogen cycles are performed and the solution is then left under vigorous stirring in a hydrogen atmosphere at atmospheric pressure at room temperature. After one hour the reaction is complete. The catalyst is filtered and the solvent is removed at reduced pressure. The product is purified by means of column chromatography (hexane-AcOEt 8:2 v/v). The pure product is obtained as a colourless oil with a yield of 93%.

### Preparation of the Compound XII

Imidazole (528 mg, 7.7 mmoles, 2.5 eq) and TBS-Cl (536 mg, 3.5 mmoles, 1.15 eq) are added, in the above order, at room temperature to a solution of the alcohol XI (1.3 g, 3.1 mmoles) in dichloromethane (35 mL). The formation of a white precipitate can be immediately noted, the reaction is performed under stirring at room temperature and is complete after 18 hours. To quench the reaction, a saturated solution of sodium bicarbonate (25 mL) is added, it is diluted with dichloromethane (20 mL), the phases are separated, the aqueous phase is extracted with dichloromethane, the re-combined organic phases are dried on magnesium sulphate and filtered, and lastly the solvent is removed at reduced pressure. The product is purified by means of column chromatography (hexane-AcOEt 9:1 v/v) and the pure product is obtained as a white solid with a yield of 95%.

### Preparation of the Compound XIII

DIBAL-H (1 M in hexane, 4.32 ml, 4.32 mmoles, 1.15 eq) is added slowly to a solution of the lactone XII (2 g, 3.76 mmoles) in dichloromethane (60 mL) cooled to −30° C. in a static argon atmosphere. Once the additions have been made, the reaction is complete after 30 minutes. To decompose the reducing agent, a saturated solution of Rochelle salts (80 mL) is added, again at −30° C., and the solution is diluted with dichloromethane; after a few minutes the dry ice and acetone bath is removed and the solution is left under vigorous stirring until the two phases can be clearly distinguished (approximately 90 minutes). The phases are separated and the aqueous phase is extracted with dichloromethane; the re-combined organic phases are dried on magnesium sulphate, the solid residue is filtered and the solvent is removed at reduced pressure. The product obtained with a quantitative yield is not purified but is used directly for the subsequent reaction.

### Preparation of the Compound XIV

The potassium tert-butylate (4 g, 35.5 mmoles, 9 eq) is added at room temperature in small portions to a suspension of (4-carboxybutyl)triphenylphosphonium bromide (8 g, 17.7 mmoles, 4.5 eq) in tetrahydrofuran (45 mL) in a static argon atmosphere; during the addition the solution heats up and takes on an orange colouring which increasingly verges on bright red. The solution is left for 30 minutes under stirring at room temperature and is then cooled to 0° C., after which the lactol XIII is added ‘via cannula’ (2 g, 3.94 mmoles) dissolved in tetrahydrofuran (20 mL); the solution turns paler, after 15 minutes the ice and water bath is removed and the solution is left under stirring at room temperature. After three hours the reaction is complete and is quenched by adding a saturated solution of ammonium chloride (100 mL) and acetic acid (2 mL, 1.05 eq with respect to the potassium tert-butylate); the solution is left under stirring for 15 minutes, then diluted with ethyl ether, the phases are separated, the aqueous phase is extracted with ethyl ether, and the re-combined organic phases are dried on magnesium sulphate, filtered and concentrated at reduced pressure. After purification by chromatography (hexane-AcOEt 8:2 v/v) the compound XIV is obtained as a colourless oil with a yield of 93%.

### Preparation of the Compound XV

HCl 1.2 N (4.5 mL) is added at room temperature to a solution of the compound XIV (2 g, 3.23 moles) in a tetrahydrofuran/water 1:1 (50 mL) mixture, the reaction is performed under vigorous stirring and is complete after approximately 18 hours. It is quenched by adding phosphate buffer (pH=6.8, 150 mL), then the organic phase is diluted with AcOEt, the two phases are separated, the aqueous phase is extracted with AcOEt, the re-combined organic phases are dried on magnesium sulphate and filtered, and lastly the solvent is removed at reduced pressure. The product is purified by means of column chromatography (pure AcOEt). The product is obtained pure as a colourless oil with a yield of 91%.

### Preparation of the Compound XVI (Latanoprost)

The enzyme Lipase Novozym 435 (500 mg) is added to a solution of XV (1 g, 2.56 mmoles) in isopropyl alcohol (10 mL). The solution is kept at 30° C. under magnetic stirring (never above 200 rpm). The reaction is complete after 18 hours. The enzyme is simply filtered and recovered, and the solvent is removed at reduced pressure. The product is purified by means of column chromatography (pure AcOEt) to give the pure product in the form of a pale yellow oil with a yield of 92%.

### Example 5

### Preparation of the Travaprost (Scheme IV)

### Preparation of the Compound XVII

Imidazole (100 mg, 1.45 mmoles, 2.5 eq) and TBS-Cl (101 mg, 0.67 mmoles, 1.15 eq) are added, in the above order, at room temperature to a solution of the alcohol IV (292 g, 0.58 mmoles) in dichloromethane (6 mL). The formation of a white precipitate can be immediately noted, the reaction is performed under stirring at room temperature and is complete after 18 hours. To quench the reaction, a saturated solution of sodium bicarbonate (12 mL) is added, it is diluted with dichloromethane, the phases are separated, the aqueous phase is extracted with dichloromethane, the re-combined organic phases are dried on magnesium sulphate and filtered, and lastly the solvent is removed at reduced pressure. The product is purified by means of column chromatography (hexane-AcOEt 9:1 v/v) and the pure product is obtained as a white solid with a yield of 87%.

### Preparation of the Compound XVIII

DIBAL-H (1 M in hexane, 402 μl, 0.402 mmoles, 1.15 eq) is slowly added to a solution of lactone XVII (214 mg, 0.35 mmoles) in dichloromethane (4 mL) and cooled to −30° C. in a static argon atmosphere. Once the additions have been made, the reaction is complete after 30 min. To decompose the reducing agent, a saturated solution of Rochelle salts (10 mL) is added, again at −30° C., and the solution is diluted with dichloromethane; after a few minutes the dry ice and acetone bath is removed and the solution is stirred vigorously until the two phases can be clearly distinguished (approximately 90 minutes). The phases are separated and the aqueous phase is extracted with dichloromethane; the re-combined organic phases are dried on magnesium sulphate, the solid residue is filtered and the solvent is removed at reduced pressure. The resulting product is a colourless oil, it is obtained with a quantitative yield and is not purified but used directly for the subsequent reaction.

### Preparation of the Compound XIX

The potassium tert-butylate (321 g, 2.86 mmoles, 9 eq) is added at room temperature in small portions to a suspension of (4-carboxybutyl)triphenylphosphonium bromide (633 mg, 1.43 mmoles, 4.5 eq) in tetrahydrofuran (7 mL) in a static argon atmosphere; during the additions the solution heats up and takes on an orange colouring which increasingly verges on bright red. The solution is left for 30 minutes under stirring at room temperature and is then cooled to 0° C., after which the lactol XVIII is added ‘via cannula’ (195 mg, 0.32 mmoles) dissolved in tetrahydrofuran (5 mL); the solution turns paler, after 15 minutes the ice and water bath is removed and the solution is left under stirring at room temperature. After three hours the reaction is complete and is quenched by adding a saturated solution of ammonium chloride (15 mL) and acetic acid (170 μl, 1.05 eq with respect to the potassium tert-butylate); the solution is left under stirring for 15 minutes and then diluted with ethyl ether, the phases are separated, the aqueous phase is extracted with ethyl ether, and the re-combined organic phases are dried on magnesium sulphate, filtered and concentrated at reduced pressure. After purification by chromatography (hexane-AcOEt 8:2 v/v) the compound XIX is obtained as a colourless oil with a yield of 96%.

### Preparation of the Compound XX

HCl 1.2 N (800 μL) is added at room temperature to a solution of the compound XIX (120 g, 0.17 mmoles) in a tetrahydrofuran/water 1:1 (10 mL) mixture, the reaction is performed under vigorous stirring and is complete after approximately 18 hours. It is quenched by adding phosphate buffer (pH=6.8, mL), then the organic phase is diluted with AcOEt, the two phases are separated, the aqueous phase is extracted with AcOEt, the re-combined organic phases are dried on magnesium sulphate and filtered, and lastly the solvent is removed at reduced pressure. The product is purified by means of column chromatography (pure AcOEt). The product is obtained pure as a colourless oil with a yield of 70%.

### Preparation of the Compound XXI (Travoprost)

The enzyme Lipase Novozym 435 (15 mg) is added to a solution of XX (30 g, 0.065 mmoles) in isopropyl alcohol (450 μL). The solution is kept at 30° C. under magnetic stirring (never above 200 rpm). The reaction is complete after 18 hours. The enzyme is simply filtered and recovered, and the solvent is removed at reduced pressure. The product is purified by means of column chromatography (pure AcOEt) to give the pure product in the form of a colourless oil with a yield of 93%.

1. A process for the preparation of prostaglandin derivatives which comprises:
(a) reacting ([3αR(3αα,4α,5β,6αα)]-(−)-5-(tert-butyldimethylsilyl)hexahydro-2-oxo-2H-cyclopenta[b]furan-4-carboxaldehyde) of formula (I) 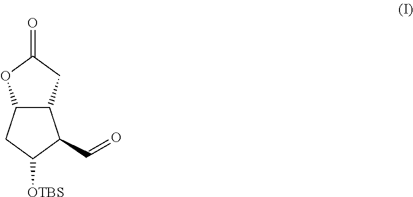
with a phosphonate of formula (II) 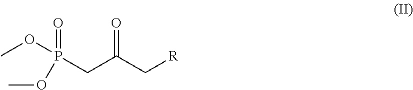
wherein R represents
a benzyl group, or
a phenoxy group, wherein the phenyl may be optionally substituted by a group selected among halogens, hydroxy derivatives, alkyls, aryls, heteroaryls and trifluoromethyl,
in presence of a strong base and a suitable solvent, to give the compound of formula (III) 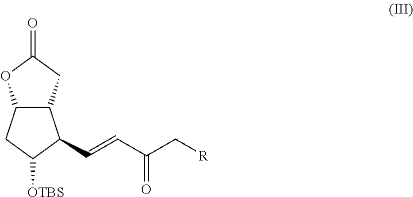
and
(b) reducing the oxo group of the side chain with an asymmetric reducing agent in presence of a suitable solvent, to give compound (IV)

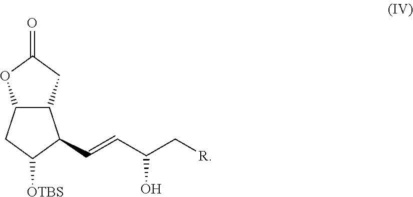

2. Process according to claim 1, characterized in that R represents a non-substituted benzyl group or a phenoxy group substituted by 3-trifluoromethyl.

3. Process according to claim 1, characterized in that the strong base used in the reaction step (a) is a hydride.

4. Process according to claim 1, characterized in that the asymmetric reducing agent is DIP-Cl.

5. Compound selected among the compounds of formula (III) 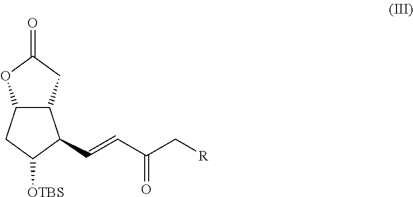
and (IV) 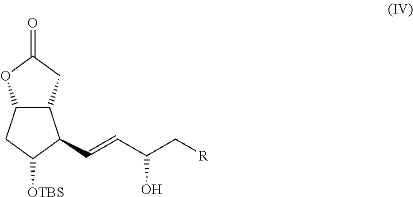
wherein R is as defined in claim 1.

6. Compound of formula (IV) as defined in claim 5, wherein R is selected among a non-substituted benzyl group or a phenoxy group substituted by a 3-trifluoromethyl.

7. Process for the preparation of bimatoprost which comprises
(c) protecting the hydroxy group of the compound of formula (IV) wherein R is a non substituted benzyl group to give the compound of formula (V) 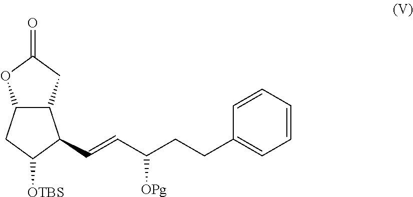
wherein Pg is a protective group, preferably TBS;
(d) reducing the oxo group to give the compound of formula (VI) 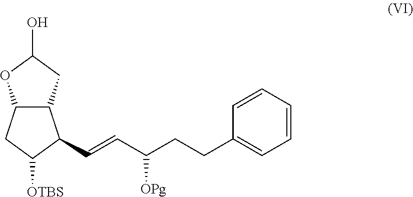
(e) reacting the compound of formula (VI) with the compound of formula:
Ph₃P═CH(CH)₃COOM
wherein M is an alkali metal, preferably potassium, to give the compound of formula (VII) 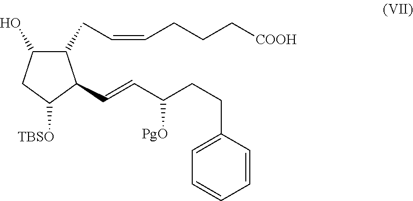
(f) esterifying the compound of formula (VII) to give the compound (VIII) 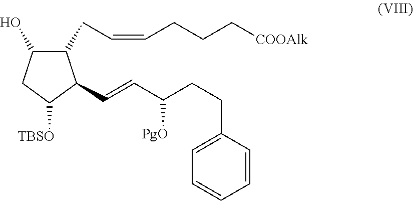
wherein Alk is the residue of a lower alkyl, preferably methyl;
(g) preparing the amide of the compound (VIII) to give the compound (IX) 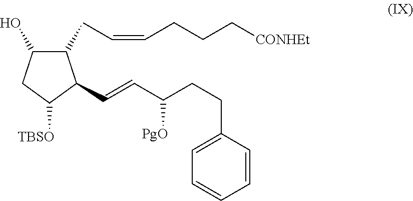
and
(h) cleaving the protecting groups from the compound (IX) to give bimatoprost of formula (X)

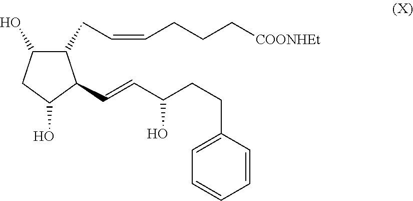

8. Process for the preparation of latanoprost which comprises
(c′) reducing the double bond of the compound of formula (IV) wherein R is a non-substituted benzyl group to give the compound of formula (XI) 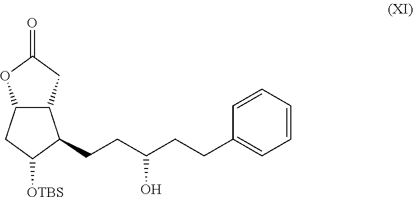
(d′) protecting the free hydroxy group of the compound of formula (XI) to give the compound of formula (XII) wherein Pg is a protective group, preferably TBS; 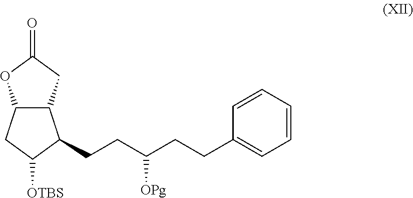
(e′) reducing the oxo group of the compound of formula (XII) to give the compound of formula (XIII) 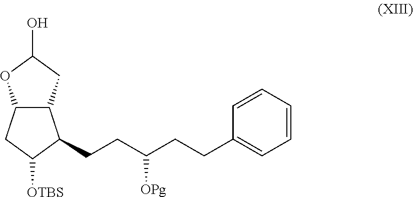
(f) reacting the compound of formula (XIII) with the compound of formula:
Ph₃P═CH(CH)₃COOM
wherein M is an alkali metal, preferably potassium, to give the compound of formula (XIV) 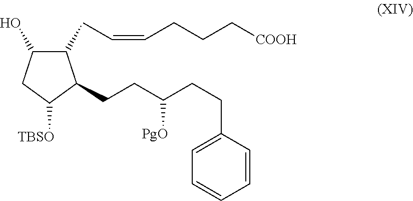
(g′) deprotecting the compound of formula (XIV) to give the compound (XV) 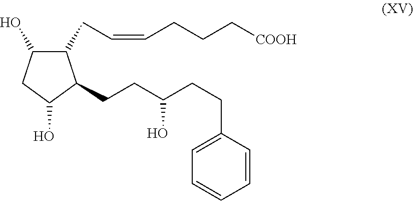
and
(h′) esterifying the compound (XV) to give latanoprost of formula (XVI)

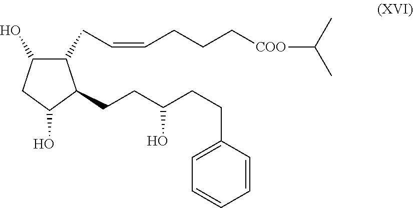

9. Process for the preparation of travoprost which comprises
(c″) protecting the free hydroxy of the compound of formula (IV) wherein R is a trifluoromethylphenoxy group to give the compound of formula (XVII) 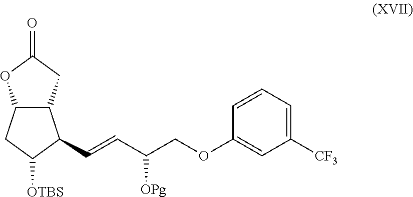
wherein Pg is a protecting group, preferably TBS;
(d″) reducing the oxo group to give the compound of formula (XVIII) 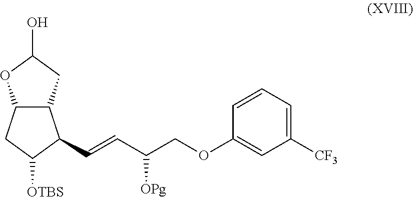
(e″) reacting the compound of formula (XVIII) with the compound of formula:
Ph₃P═CH(CH)₃COOM
wherein M is an alkali metal, preferably potassium, to give the compound of formula (XIX) 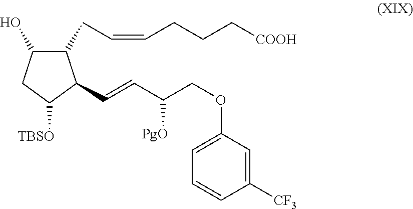
(f″) deprotecting the compound of formula (XIX) to give the compound (XX) 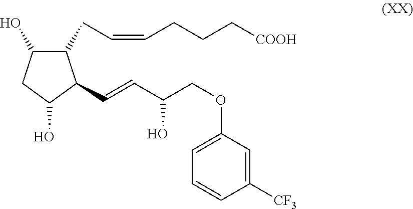
(g″) esterifying the compound (XX) to give travoprost of formula (XXI) 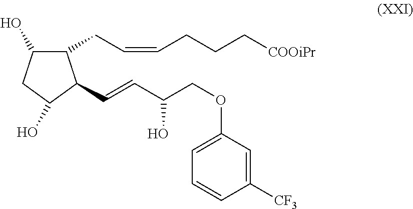
wherein iPr is an isopropylic residue.

10. Compound independently selected among the compounds of formula (V), (VI), (VII), (IX), (XI), (XII), (XIII), (XIV), (XV), (XVII), (XVIII), (XIX) and (XX), as defined in claim 7.

11. Compound of formula 10 wherein:
Pg, if any, is TBS; and
R, if any, is selected between a non-substituted benzyl group or a phenoxy group substituted with a 3-trifluoromethyl group.

12. Use of the compound of formula (IV) according to claim 5 for the preparation of prostaglandin derivatives.
